(19) United States
(12) Patent Application Publication
(10) Pub. No.: US 2006/0252997 A1
(43) Pub. Date: Nov. 9, 2006

(54) Medical port device, kit and associated method

(75) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(73) Assignee: WILK PATENT, LLC, New York, NY (US)

(21) Appl. No.: 11/388,651

(22) Filed: Mar. 24, 2006

Related U.S. Application Data
(60) Provisional application No. 60/674,040, filed on Apr. 22, 2005.

(51) Int. Cl. A61F 11/00; A61B 1/32

(52) U.S. Cl. 600/237

(57) ABSTRACT

In a trans-organ or flexible endoscopic procedure, a surgical device including an annular frame member made of substantially rigid material and at least one cushion element connected to the frame member and disposed along an outer surface thereof on at least two opposing sides of the frame member is disposed in a patient's mouth between upper and lower teeth thereof so that at least some of the teeth are temporarily embedded in the cushion element and so that a hole in the frame member enables access to the patient's esophagus. Subsequently an elongate flexible shaft of a surgical instrument is inserted into the patient's esophagus and stomach through the hole on the frame member.

### CROSS-REFERENCE TO RELATED APPLICATION

This application claims the benefit of U.S. Provisional Patent Application No. 60/674,040 filed Apr. 22, 2005.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insufflated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduce trauma to the individual even more than laparoscopic procedures. Hospital convalescence stays are even shorter. There are some potential problems with the procedures. One such problem is that, in accessing the patient's abdominal cavity via the stomach and mouth, that the unconscious patient may close his or her jaws about the delicate operating instruments or even more delicate fingers of the operating surgeon.

### OBJECTS OF THE INVENTION

It is an object of the present invention to provide improvements on the afore-described surgical procedures.

It is another object of the present invention to provide a method and/or an associated device for protecting instruments and fingers from inadvertent damage owing to unconscious biting action of the patient, where instruments are passed through the mouth and stomach into the patient's abdominal cavity.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A medical port device in accordance with the present invention comprises an annular frame member made of substantially rigid material, and at least one cushion element connected to the frame member and disposed along an outer surface thereof on at least two opposing sides of the frame member. The frame member and the cushion element(s) are configured so as to fit inside the mouth of a human being so as to hold the mouth open while enabling the passage of a medical instrument through the mouth.

Pursuant to another feature of the present invention, the cushion element includes a gel material. The gel material may be disposed in a flexible pouch or bag. This prevents the gel mass or portions thereof from inadvertently moving into the trachea or esophagus of the patient.

The gel material may be of the type that is used by dentists in forming molds of patient's teeth. In that case, the pouch or bag could be eliminated, inasmuch as the natural cohesiveness and stickiness of the gel material will militate against inadvertent migration of gel particles or masses.

The cushion element may be one of a pair of cushion elements each including a quantity of gel disposed in a pouch or bag. The cushion elements are, in that case, disposed on opposite sides of the frame member for engaging and receiving the teeth of the opposing jaws.

Pursuant to a further feature of the present invention, the frame member is formed with a circumferential groove along the outer surface, with the cushion element being at least partially disposed in the groove. As indicated above, the cushion element may include a gel material held in the groove by virtue of a natural adhesiveness of the gel material. Alternatively, the gel material may be disposed in a pouch or bag. The pouch or bag may be attached to the frame member, for instance, to the surface of the groove, by adhesive.

A surgical method in accordance with the present invention comprises providing a surgical port device including an annular frame member made of substantially rigid material, and at least one cushion element connected to the frame member and disposed along an outer surface thereof on at least two opposing sides of the frame member. The method further comprises disposing the surgical device in a patient's mouth between upper and lower teeth thereof so that at least some of the teeth are temporarily embedded in the cushion element and so that a hole in the frame member enables access to the patient's esophagus. Subsequently, an elongate flexible shaft of a surgical instrument is inserted into the patient's esophagus and stomach through the hole in the frame member of the port device.

Where the cushion includes gel material, the providing of the surgical device may include depositing the gel material on the frame member. Thus, the gel material may be applied to the frame member in the operating room. Alternatively, the gel may be disposed on the frame member during the manufacturing process and packaged in an assembled device with the frame member.

Where the cushion includes gel material disposed in a bag or pouch, the providing of the surgical device may include depositing the bag or pouch on the frame member. Again, the cushion pouches or bags may be applied to the frame member in the operating room. Alternatively, the gel-filled pouches or bags may be disposed on the frame member during the manufacturing process and packaged in an assembled device with the frame member.

### BRIEF DESCRIPTION OF THE DRAWING

FIG. 1 is a schematic perspective view of a frame member of a port device in accordance with the present invention.

FIG. 2 is a schematic perspective view of a port device in accordance with the present invention, including the frame member of FIG. 1.

### DETAILED DESCRIPTION

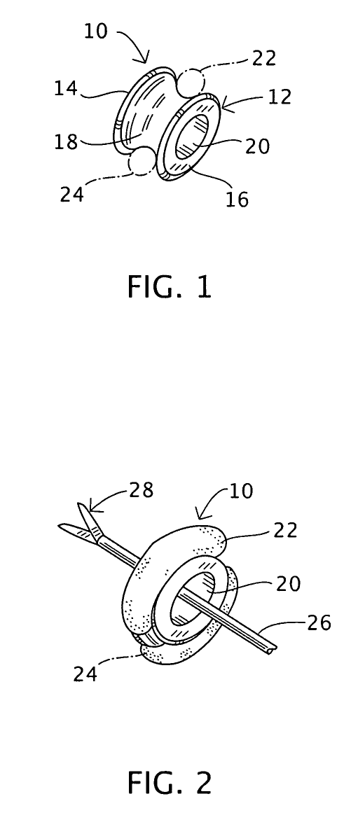

As illustrated in FIG. 1, a medical port device 10 comprises an annular frame member 12 made of substantially rigid material. Frame member 12 includes a pair of flanges or rims 14 and 16 flanking a circumferential groove 18 that may take the form of a concave toroidal section. Frame member 12 defines a circular hole or opening 20 that constitutes a port for the passage of flexible endoscopic instruments. Multiple such instruments may be inserted in a trans-organ surgical procedure through a patient's mouth, along the esophagus, into the stomach and through an artificial opening or perforation formed in the stomach wall, as described in U.S. Pat. Nos. 5,297,536 and 5,458,131.

As illustrated in FIG. 2, port device 10 further includes a pair of cushion elements 22 and 24 each partially disposed in groove 18 on opposite sides of frame member 12 and along an outer surface thereof. Cushion elements 22 and 24 each include a mass of gel material such as that used by dentists to form molds of a patient's teeth. The masses of gel material may be disposed in respective flexible pouches or bags (not separately labeled) that form envelopes adhering to the gel masses. The pouches or bags prevent the gel masses or portions thereof from inadvertently moving into the trachea or esophagus of the patient.

In use of port device 10, frame member 12 is disposed in a patient's mouth between the upper and lower teeth. The gel material is forced around the teeth so that the upper and the lower teeth are embedded in cushions 22 and 24, respectively. Subsequently, an elongate flexible shaft 26 of a surgical instrument 28 is inserted into the patient's esophagus and stomach through hole or opening 20.

One or more surgical instruments including instrument 28 may be packaged together with medical port device 10 in a kit for facilitating commercial distribution and use in surgical procedures. Shaft 26 is long enough to extend to a patient's stomach (and beyond) when inserted through the mouth.

Although the invention has been described in terms of particular embodiments and applications, one of ordinary skill in the art, in light of this teaching, can generate additional embodiments and modifications without departing from the spirit of or exceeding the scope of the claimed invention. For example, instead of a gel material, the cushion or cushions may comprise a polymeric foam material. The foam material may be stiff and deformable or resilient.

In addition, it is possible for a port device in accordance with the invention to have a single cushion element that is either annular or C-shaped.

The present invention may be used during conventional endoscopic upper GI procedures.

Accordingly, it is to be understood that the drawings and descriptions herein are profferred by way of example to facilitate comprehension of the invention and should not be construed to limit the scope thereof.

What is claimed is:

1. A medical port device comprising:
an annular frame member made of substantially rigid material; and
at least one cushion element connected to said frame member and disposed along an outer surface thereof on at least two opposing sides of said frame member.

2. The device defined in claim 1 wherein said cushion element includes a gel material.

3. The device defined in claim 2 wherein said cushion element further includes a flexible pouch or bag, said gel being disposed in said pouch or bag.

4. The device defined in claim 3 wherein said cushion element is one of a pair of cushion elements each including a quantity of gel disposed in a pouch or bag, said cushion elements being disposed on opposite sides of said frame member.

5. The device defined in claim 1 wherein said frame member is formed with a circumferential groove along said outer surface, said cushion element being at least partially disposed in said groove.

6. The device defined in claim 5 wherein said cushion element includes a gel material.

7. The device defined in claim 6 wherein said gel material is held to said frame member by virtue of a natural adhesiveness of the gel material.

8. The device defined in claim 6 wherein said cushion element is one of a pair of cushion elements each in the form of a mass of said gel material, said cushion elements being disposed on opposite sides of said frame member.

9. The device defined in claim 1 wherein said frame member and said cushion element are configured so as to fit inside the mouth of a human being so as to hold the mouth open while enabling the passage of a medical instrument through the mouth.

10. A surgical method comprising:
providing a surgical port device including an annular frame member made of substantially rigid material, and at least one cushion element connected to said frame member and disposed along an outer surface thereof on at least two opposing sides of said frame member;
disposing said surgical device in a patient's mouth between upper and lower teeth thereof so that at least some of said teeth are temporarily embedded in said cushion element and so that a hole in said frame member enables access to the patient's esophagus; and
subsequently inserting an elongate flexible shaft of a surgical instrument into the patient's esophagus through said hole.

11. The method defined in claim 10 wherein said cushion includes gel material, the providing of said surgical device including depositing said gel material on said frame member.

12. The method defined in claim 10 wherein said cushion includes gel material disposed in a bag or pouch, the providing of said surgical device including depositing said bag or pouch on said frame member.

13. A surgical kit comprising:
a surgical instrument having an elongate flexible shaft;
an annular frame member made of substantially rigid material; and
at least one cushion element connectable to said frame member along an outer surface thereof on at least two opposing sides of said frame member.

14. The kit defined in claim 13 wherein said cushion element includes a gel material.

15. The kit defined in claim 14 wherein said cushion element further includes a flexible pouch or bag, said gel being disposed in said pouch or bag.

16. The kit defined in claim 13 wherein said frame member is formed with a circumferential groove along said outer surface, said cushion element being at least partially disposed in said groove.

17. The kit defined in claim 13 wherein said frame member and said cushion element are configured so as to fit inside the mouth of a human being so as to hold the mouth open while enabling the passage of a medical instrument through the mouth.
